(19) United States
(12) Patent Application Publication
(10) Pub. No.: US 2012/0265048 A1
(43) Pub. Date: Oct. 18, 2012

(54) SYSTEM FOR PROVIDING MAGNETIC, LIGHT AND CRYSTAL ENERGY THERAPY

(75) Inventors: TERRYL E. BIGGS, GARLAND, TX (US); KEVIN FRANK, WILEY, TX (US); FRANK SKAGGS, DALLAS, TX (US); TAMMY J. SMITH LAHUTSKY, PLANO, TX (US); TIMOTHY B. SMITH, DALLAS, TX (US)

(73) Assignee: AVAZZIA, INC., DALLAS, TX (US)

(21) Appl. No.: 13/446,167

(22) Filed: Apr. 13, 2012

Related U.S. Application Data
(60) Provisional application No. 61/475,707, filed on Apr. 15, 2011.

(51) Int. Cl. A61N 2/02; A61N 2/04; A61N 5/06; A61N 1/00; A61B 5/05; A61N 5/067

(52) U.S. Cl. 600/409; 600/13; 600/14

(57) ABSTRACT

A system for providing treatment signals to a body of an individual includes a magnetic field measurement circuitry for measuring the magnetic field of the body of the individual and providing feedback information responsive thereto. A user interface displays the feedback information and receives a control input. A plurality of energy treatment circuitries each provides treatment energy to the body of the individual responsive to at least one control signal. An application interface applies the treatment energy to the body of an individual. A control block generates the at least one control signal responsive to the control input.

### CROSS-REFERENCE TO RELATED APPLICATIONS

This application claims benefit of U.S. Provisional Application No. 61/475,707, filed on Apr. 15, 2011, and entitled LIGHT SABER, the specification of which is incorporated herein by reference.

### TECHNICAL FIELD

The present invention relates to a system for treating injuries, and more particularly, to a system for treating injuries using various combinations of laser light energy, LED energy, magnetic energy, electro-stimulation energy and crystal energy.

### BACKGROUND

Traditional medical, homeopathic, oriental and alternative healthcare treatments utilize various technologies and techniques for pain management, accelerated healing processes and diagnostic processes. These can include things such as drug therapy, manipulation therapy, biofeedback therapy, acupuncture, chi therapy, electro-stimulation therapy, magnetic field therapy, laser therapy, light therapy and crystal therapy. Each of these techniques are utilized individually in an attempt to provide various types of pain management, accelerated healing or diagnostic techniques.

The problem with the solutions associated with these types of therapies is that the products are normally implemented within bulky systems that are very impractical for portable usage. These therapies and technologies while implementable within a clinical or doctor's office are not practical for home use. Additionally, these various types of therapies will often fail to provide objective evidence of the efficacy of the process or treatment. Therefore, there is a need for some manner for the treatment of pain management, accelerated healing or diagnostic techniques that overcome these problems within a portable system that is easily utilizable by, for example, a home user.

### SUMMARY

The present invention as disclosed and described herein, in one aspect thereof, comprises a system for providing treatment signals to a body of an individual and includes a magnetic field measurement circuitry for measuring the magnetic field of the body of the individual and providing feedback information responsive thereto. A user interface displays the feedback information and receives a control input. A plurality of energy treatment circuitries each provides treatment energy to the body of the individual responsive to at least one control signal. An application interface applies the treatment energy to the body of an individual. A control block generates the at least one control signal responsive to the control input.

### BRIEF DESCRIPTION OF THE DRAWINGS

For a more complete understanding, reference is now made to the following description taken in conjunction with the accompanying Drawings in which:

FIG. 1 is a basic block diagram of the system for providing pain management, accelerated healing and diagnostic treatment;

FIG. 2 is a more detailed block diagram of the system of FIG. 1;

FIG. 3a illustrates a stacked, parallel configuration of windings surrounding a crystal;

FIG. 3b illustrates a serial configuration of windings surrounding a crystal;

FIG. 3c illustrates a first scalar configuration of windings surrounding a crystal;

FIG. 3d illustrates a second scalar configuration of windings surrounding a crystal;

FIG. 3e illustrates a third scalar configuration of windings surrounding a crystal;

FIG. 3f illustrates a magnetic polarity configuration of windings surrounding a crystal; and

FIG. 4 illustrates various control inputs provided to a crystal to provide a different output.

### DETAILED DESCRIPTION

Referring now to the drawings, wherein like reference numbers are used herein to designate like elements throughout, the various views and embodiments of a system for providing magnetic, light, electro-stimulation and crystal energy therapy are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

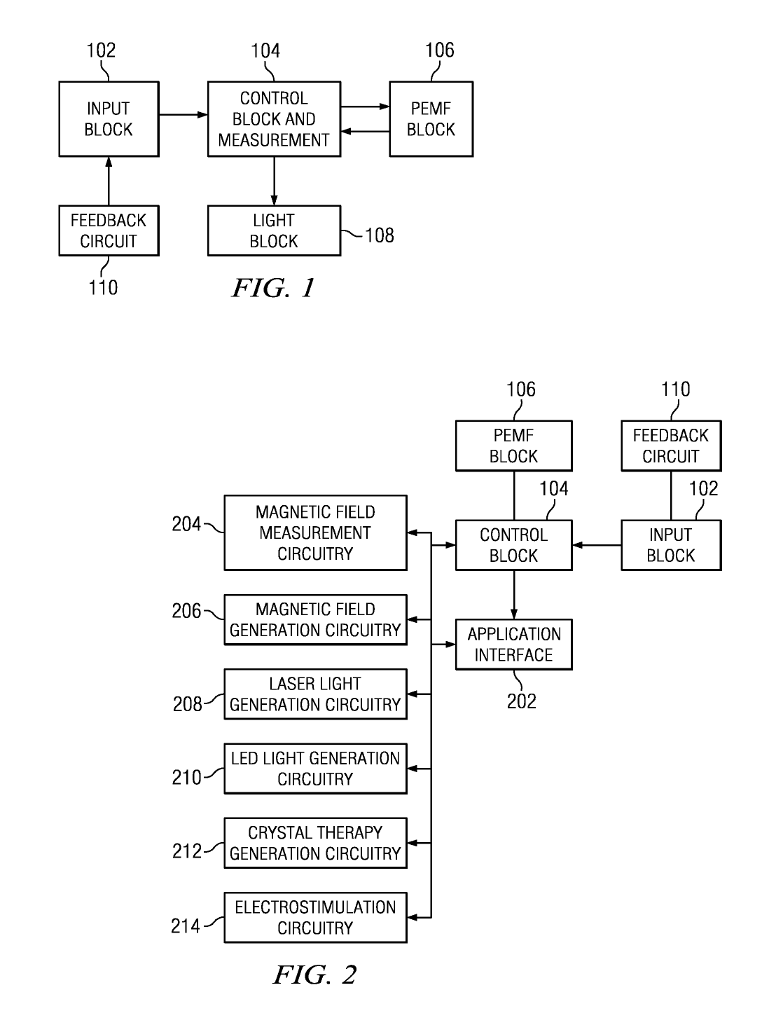

Referring now to the drawings, and more particularly to FIG. 1, there is illustrated a general block diagram of the system for providing treatment for pain management, accelerated healing and diagnostic analysis. As described previously, present systems for providing treatment for pain management, accelerated healing or diagnostic purposes utilize bulky system components for providing each of these treatment processes. The present system, as illustrated with respect to FIG. 1, comprises a system allowing the integration of electro-stimulation therapy, magnetic field therapy, laser therapy, light therapy and crystal therapy into a single handheld device. The system will additionally provide a mechanism for the assessment of anomalies in the magnetic field of an individual's body and allow the tracking of the magnetic field before therapy, during therapy and after therapy to provide real-time feedback to the system. Thus, a user can receive objective, measurable evidence of the benefits of various types of treatment or combinations thereof being provided to an individual. The system enables the measurement of variations in the magnetic field of the human body for assessment of anomalies and assessment of changes resulting from various therapies. This can provide objective evidence of problems and changes in the body arising from variously applied therapies.

The system comprises an input block 102 for monitoring of feedback with respect to treatments being applied to an individual. The control block and measurement block 104 control the various parameters applying light, magnetic, electro-stimulation and crystal therapy to a patient.

The input block 102 connects the device to a feedback circuit 110 that provides a microcurrent biofeedback electro-stimulation signal or other type of feedback control signal to the control block 104. The feedback circuit 110 may also provide some other type of frequency modulation control circuitry for providing the feedback signals to the input block 102. The control block 104 controls the manner for increasing or decreasing the field strength or changing the polar orientations of the various magnetic, light and crystal therapies being applied by the device. The measurement circuitry of the control block and measurement block 104 provide the measurement of the detected magnetic fields detected through the PEMF block 106 to quantify changes detected in the body field of a patient.

The PEMF block 106 is used for controlling the magnetic and crystal therapy applications to the body of a patient and for detecting the magnetic field changes caused by the various therapies that are applied to a patient. The light block 108 controls and drives the various light sources that are being used to provide laser and light therapy to an individual patient. The light block 108 provides the drive control and light sources that project light at various wavelengths of visible light and non-visible light spectrums around and/or through the magnetic and/or crystal treatment therapies. Application of light sources through the light block 108 enable the light to be applied around and/or through magnetic and/or crystal therapies to multiply the effects provided by each therapy.

Referring now to FIG. 2, there is illustrated a more detailed block diagram of the system of FIG. 1. This figure more fully illustrates the various therapy control circuitries that are controlled via the control block 104 for application to the body of a patient through the application interface 202. As before, the control block 104 receives feedback through input block 102 provided from the feedback circuit 110 and selects the therapy circuits needed to apply a selected therapy to a patient. The PEMF block provides magnetic field readings to the control block 104 to indicate the benefits being provided by the application of various treatment therapies to a patient. The therapy feedback circuitry consists of the magnetic field measurement circuitry 204 that enables the magnetic field of a person's body to be detected and provide feedback information to the control block 104 providing for the control of various therapies applied to a patient's body based upon these objective measurements. The magnetic field sensing circuitry 204 may consist in one embodiment of a faraday pickup coil that may detect the differences in the body's magnetic field strength and differences in the direction of the magnetic pole field. The magnetic field sensing circuitry 204 can determine if the body's magnetic field is north (positive voltage) or south (negative voltage). The magnetic field sensing circuitry 204 can additionally measure the amplitude of the magnetic field strength to determine the amplitude and polarity of the body voltage. The integration of the magnetic field sensing circuitry 204 allows for the measurement and quantification of magnetic body field changes within a patient. Magnetic field measurement circuitry 204 enables the measurements of variations in the magnetic field of the human body to enable the assessment of anomalies occurring within the body and to assess changes resulting from the various applied combination of therapies. This enables the provision of objective evidence of problems and changes resulting from the therapies being applied to the body of a patient.

The magnetic field generation circuitry 206 provides for the generation of a magnetic field at a desired level for application to the body of a patient through the application interface 202. The magnetic field generation circuitry 206 uses various windings, orientations and wire gauges to implement a desired field direction and strength of the magnetic field being applied to the body of a patient through the application interface 202. In one embodiment, the magnetic field generation circuitry 206 may comprise a Tesla QI device having the ability to provide different magnetic field strengths including the ability to generate a scalar magnetic field. The magnetic field generation circuitry 206 will also have the ability to switch between a north magnetic pole and a south magnetic pole being projected from the circuitry. The magnetic field generation circuitry 206 allows for the application of a damped sinusoidal magnetic field and through the control block, the strength of the field may be increased or decreased and the polar orientation of the field being applied to a patient may also be controlled.

The laser light generation circuitry 208 utilizes laser light generation components to generate a laser for application to the body of a patient. Similarly, the LED light generation circuitry 210 utilizes LED light circuitry for providing LED lights for application to the body of a patient through the application interface 202. Each of the laser light generation circuitry 208 and LED light generation circuitry 210 can generate lights at a variety of frequency and color levels enabling the application of, for example, ultraviolet light, violet light, blue light, orange light, yellow light, green light, red light and infrared light to the body of a patient through the application interface 202. The laser light sources applied through the laser light circuitry 210 can increase the depth of the therapy within a patient while the LED light sources can provide a convenient achievement of various wavelengths of therapy.

The crystal therapy generation circuitry 212 utilizes various combinations of crystals and orientations and compositions of the crystals to achieve variations in results that are applied to a patient through the application interface 202. The crystal therapy generation circuitry 212 comprise a crystal that is used a core of a winding. The crystals are then excited by electro magnetic and/or optical light energy or magnetic energy patterns. The applied magnetic field will modify the electrical structure of the crystal slightly (to a small degree). The crystal acts as an extremely high-Q capacitor that slowly leaks off its energy.

The crystal therapy generation circuitry 212 can be controlled in a number of manners. The frequency of the output of the crystal therapy generation circuitry 212 may be modulated. The intensity of the output of the crystal therapy generation circuitry 212 can be modulated or the power level of the output can be varied. The frequency of the output (in Hz) can be modulated or varied. The output can be provided as a pulsed high voltage sinusoidal signal. Also, the polarization of the output can be rotated to vary the output signal to provide different effects. Additional the light wavelength frequency of the output can be varied by using different light colors, different light wavelength frequencies of light within both the visible and non-visible ranges (e.g., infrared and ultra-violet light). The control circuitry may also control the manner in which the crystals of the crystal therapy generation circuitry are excited. A modified excitation of the crystal may be achieved by various combinations of control of: a) the presence and type of the magnetic field, b) modulation of the magnetic field sequence, and c) the optical color light frequencies.

The crystal therapy generation circuitry 212 may have a number of different combinations of the windings that surround a variety of types of crystal cores. The crystal cores can comprise a variety of shapes including a tube, a sphere, a star shape or even a crystal configuration of a tube holding a number of broken or smaller crystals. Various combinations of types of crystals may also be used to alter the effect to the body of a patient.

The light frequencies applied to the crystals may be applied to the crystals in various combinations including either before the crystal so that the light shines through the crystal or after the crystal so that the light wave frequency is combined with the electro-magnetic frequency.

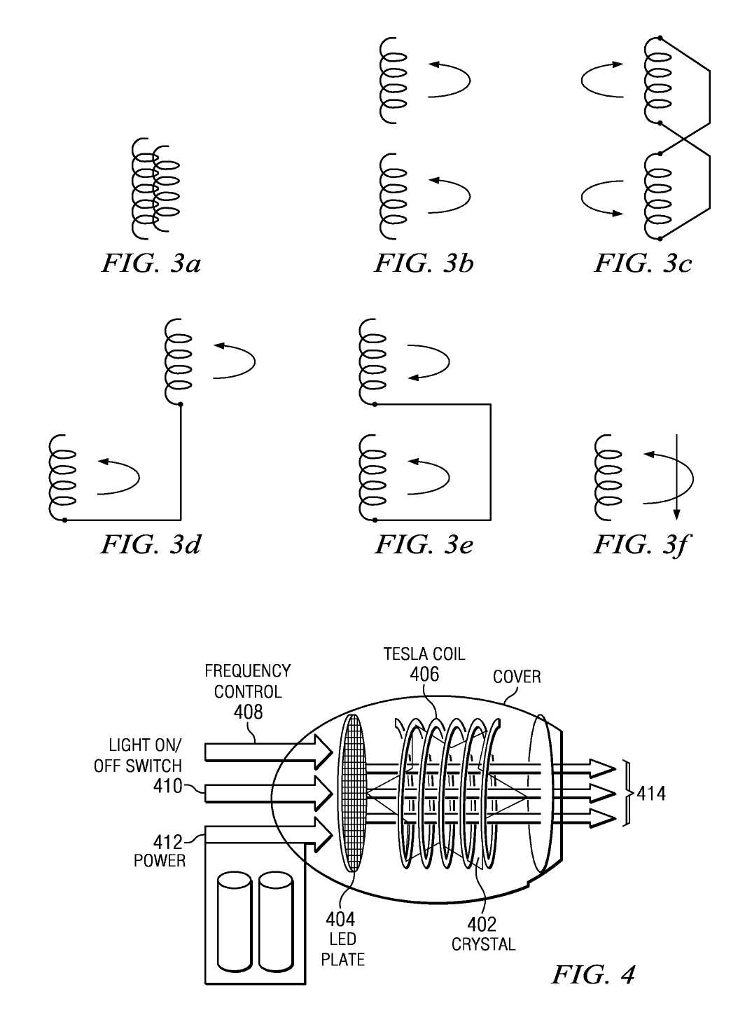

The crystal therapy generation circuitry 212 may also include a number of different patterns for the windings surrounding the crystal core. If at least two windings are used they may use a stacked, parallel combination (FIG. 3a); a serial combination where the polarity of each winding is in the same direction (FIG. 3b); a scalar combination where the windings are shorted together and have opposite polarities (FIG. 3c); a scalar combination where the windings are connected at one end and have polarities in the same direction (FIG. 3d); a scalar combination where the windings are connected at one end and have polarities in the opposite directions (FIG. 3e); a magnetic polarity configuration where the poles of the windings may be periodically switched (FIG. 3f).

Thus, to summarize the three types of wire connection configurations for the windings include a parallel connection, a serial connection and a reverse connection. The windings may have two directions of polarity, a north pole or a south pole. The described configurations use two coils on the crystals but other numbers of crystal are possible. Various combinations of these factors may be used to achieve different treatment affects. The different variations are used because cells have been found to have minute fibers that act like antennas. Some minute magnetic fields have different effects than stronger magnetic fields that would saturate these minute fiber antennas.

Finally, the electro-stimulation circuitry 214 may be utilized to provide various electro-stimulation therapies to the body of a patient through the application interface 202. The electro-stimulation circuitry 214 may include components similar to those described with respect to U.S. patent application Ser. No. 11/747,458, filed May 11, 2007, which is incorporated herein by reference.

The control block 104 through the application interface 202 enables various combinations of magnetic treatment, laser light treatment, LED light treatment, crystal therapy treatment and electro-stimulation treatment to be applied to the body of a patient. In various embodiments, either singular ones of these treatments may be applied to the body of a patient or any combinations thereof may also be utilized. Feedback to the control block 104 is provided from the magnetic field measurement circuitry 204, which measures the effects of the various treatment combinations upon the body of a patient and enables a user of the device to determine which treatment or treatments are most positively affecting the patient and adjust the combination of therapies to achieve optimal results.

The system illustrated in FIG. 2 provides for a small lightweight and portable system capable of being used within the home. The circuitry used therein provides for low power consumption enabling battery operation. By integrating the magnetic field devices, light devices and crystal devices with the electro-stimulation circuitry, the device may increase the impact of various therapies compared to stand alone devices which only comprise individual ones of these various types of therapies. Application of light sources such that the light is applied in around and/or through the magnetic/crystal integration effects can have an impact upon the benefits of the applied therapy.

Referring now to FIG. 4, there is illustrated how a crystal 402 may have different inputs applied thereto to provide varying outputs. The crystal 402 has light applied thereto by an LED plate 404 and electrical energy and/or magnetic energy applied thereto by a Tesla Coil 406. The amount of energies applied to the crystal can be controlled by a frequency control input 408 controlling the frequency of the light, electricity or magnetic field, by a light on/off control signal 410 for controlling the LED plate 404 and a power control signal 412. The output energies 414 applied to a patient are varied by different combination of the control signals 408, 410, 412 being applied to the crystal 402.

It will be appreciated by those skilled in the art having the benefit of this disclosure that this system for providing magnetic, light and crystal energy therapy provides an improved manner for providing these treatment therapies to a patient. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A system for providing treatment signals to a body of an individual, comprising:
magnetic field measurement circuitry for measuring the magnetic field of the body of the individual and providing feedback information responsive thereto;
a user interface for displaying the feedback information and receiving a control input;
a plurality of energy treatment circuitries each providing a treatment energy to the body of the individual responsive to at least one control signal;
an application interface for applying the treatment energy to the body of an individual; and
a control block for generating the at least one control signal responsive to the control input.

2. The system of claim 1, wherein one of the plurality of treatment energy circuitries further comprises:
magnetic field generation circuitry for generating a magnetic field for application to the body of the individual as the treatment energy; and
a second energy treatment circuitries for applying the treatment energy to the body of the individual, the second energy treatment circuitries including at least one of laser light generation circuitry for generating laser light for application to the body of the individual as the treatment energy, LED light energy circuitry for generating LED light for application to the body of the individual as the treatment energy, crystal energy generation circuitry for generating crystal energy for application to the body of the individual as the treatment energy and electro-stimulation circuitry for applying electrical energy to the body of the individual as the treatment energy.

3. The system of claim 2, wherein the magnetic field generation circuitry further comprises a Tesla QI device.

4. The system of claim 2, wherein the magnetic field generation circuitry varies a strength of the magnetic field and a polarity of the magnetic field responsive to the at least one control signal.

5. The system of claim 2, wherein laser light generation circuitry generates the laser light at a variety of frequencies responsive to the at least one control signal.

6. The system of claim 2, wherein the laser light generation circuitry generates the laser light at a variety of color levels responsive to the at least one control signal.

7. The system of claim 2, wherein LED light generation circuitry generates the LED light at a variety of frequencies responsive to the at least one control signal.

8. The system of claim 2, wherein the LED light generation circuitry generates the LED light at a variety of color levels responsive to the at least one control signal.

9. The system of claim 1, wherein the magnetic field measurement circuitry comprises a faraday pickup coil.

10. A system for providing treatment signals to a body of an individual, comprising:
magnetic field measurement circuitry for measuring the magnetic field of the body of the individual and providing feedback information responsive thereto;
a user interface for displaying the feedback information and receiving a control input;
magnetic field generation circuitry for generating a magnetic field for application to the body of the individual as treatment energy responsive to at least one control signal;
laser light generation circuitry for generating laser light for application to the body of the individual as the treatment energy responsive to at least one control signal;
LED light energy circuitry for generating LED light for application to the body of the individual as the treatment energy responsive to at least one control signal;
crystal energy generation circuitry for generating crystal energy for application to the body of the individual as the treatment energy responsive to at least one control signal;
electro-stimulation circuitry for applying electrical energy to the body of the individual as the treatment energy responsive to at least one control signal;
an application interface for applying the treatment energy to the body of an individual; and
a control block for generating the at least one control signal responsive to the control input.

11. The system of claim 10, wherein the magnetic field generation circuitry further comprises a Tesla QI device.

12. The system of claim 10, wherein the magnetic field generation circuitry varies a strength of the magnetic field and a polarity of the magnetic field responsive to the at least one control signal.

13. The system of claim 10, wherein laser light generation circuitry generates the laser light at a variety of frequencies responsive to the at least one control signal.

14. The system of claim 10, wherein the laser light generation circuitry generates the laser light at a variety of color levels responsive to the at least one control signal.

15. The system of claim 10, wherein LED light generation circuitry generates the LED light at a variety of frequencies responsive to the at least one control signal.

16. The system of claim 10, wherein the LED light generation circuitry generates the LED light at a variety of color levels responsive to the at least one control signal.

17. The system of claim 10, wherein the magnetic field measurement circuitry comprises a faraday pickup coil.

18. The system of claim 10, wherein the control block controls the application interface to provide a selected combination of at least two of the magnetic field, the laser light, the LED light, the crystal energy and the electrical energy.

19. A method for providing treatment signals to a body of an individual, comprising:
applying a plurality of treatment energies to the body of a patient responsive to a control input;
measuring the magnetic field of the body of the individual and providing feedback information responsive thereto;
displaying the feedback information on a user interface; and
receiving the control input from the user interface.

20. The method of claim 19, wherein the step of applying further comprises:
generating a magnetic field for application to the body of the individual as the treatment energy; and
applying at least one second treatment energy to the body of the individual, the second energy treatment circuitries including at least one of laser light for application to the body of the individual as the second treatment energy, LED light for application to the body of the individual as the second treatment energy, crystal energy for application to the body of the individual as the second treatment energy and electrical energy for application to the body of the individual as the second treatment energy.

21. The method of claim 20, wherein the step of generating the magnetic field further comprises varying a strength of the magnetic field and a polarity of the magnetic field responsive to the at least one control signal.

22. The method of claim 20, wherein the step of applying at least one second energy treatment further comprises generating the laser light at a variety of frequencies responsive to the at least one control signal.

23. The method of claim 20, wherein the step of applying at least one second energy treatment further comprises generating the laser light at a variety of color levels responsive to the at least one control signal.

24. The method of claim 20, wherein step of applying at least one second energy treatment further comprises generating the LED light at a variety of frequencies responsive to the at least one control signal.

25. The method of claim 20, wherein the step of applying at least one second energy treatment further comprises generating the LED light at a variety of color levels responsive to the at least one control signal.
